(19) United States
(12) Patent Application Publication
(10) Pub. No.: US 2016/0120848 A1
(43) Pub. Date: May 5, 2016

(54) SPECIFIC CANCER TREATMENT REGIMES WITH GANETESPIB

(71) Applicant: SYNTA PHARMACEUTICALS CORP., Lexington, MA (US)

(72) Inventor: Vojo Vukovic, Winchester, MA (US)

(21) Appl. No.: 14/892,761

(22) PCT Filed: May 20, 2014
(86) PCT No.: PCT/US2014/038796

§ 371 Date: Nov. 20, 2015

Related U.S. Application Data
(60) Provisional application No. 61/925,027, filed on Jan. 8, 2014.
(60) Provisional application No. 61/825,566, filed on May 21, 2013.

(51) Int. Cl. A61K 31/4196; A61K 33/24; A61K 31/704; A61K 31/519; A61K 31/675; A61K 31/337; A61K 31/282

(52) U.S. Cl. CPC A61K 31/4196; A61K 31/337; A61K 33/24; A61K 31/282; A61K 31/519; A61K 31/675; A61K 31/704

(57) ABSTRACT

Methods of treating certain types of cancer with ganetespib are disclosed. Also provided are methods of treating certain types of cancer with ganetespib in combination with a taxane derivative, and a platinum-containing anticancer agent. Also provided are methods of treating certain types of cancer with p53 mutation by a combination of ganetespib with a taxane derivative, and a platinum-containing anticancer agent. Also provided are methods of treating certain types of cancer with ganetespib in combination with a platinum-containing anticancer agent, and an antimetabolite. Also provided are methods of treating certain types of cancer with ganetespib in combination with a taxane derivative, an anthracycline derivative, and an alkylating antineoplastic agent. Also provided is a pharmaceutical composition comprising ganetespib and one or more other anticancer agents as described above.

### CROSS-REFERENCE TO RELATED APPLICATIONS

This application claims the benefit of priority to U.S. Provisional Patent Application No. 61/925,027, filed on Jan. 8, 2014, and 61/825,566, filed on May 21, 2013. The contents of each of these applications are incorporated herein by reference.

### BACKGROUND OF THE INVENTION

Cancer is a group of diseases characterized by dysregulation of cell differentiation and proliferation and, in advanced stages, spread to other areas of the body including vital organs and bone. If not brought under control, these diseases can be fatal.

Through advancements in detection, surgery and therapeutic options, especially in the area of targeted therapies, patients' prognoses are generally improving, and 5-year survival rates for a number of cancers are rising. Nevertheless, the room for continued improvement in treatment options is vast: the American Cancer Society estimates approximately 1.4 million new cases of cancer will be diagnosed in the US this year, with 564,830 cancer-related deaths in 2006 in the US, and about 10 times this number worldwide (cancer.org).

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is unlikely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer. Examples of Hsp90 client proteins that have been implicated in the progression of cancer are described below.

Her2 is a transmembrane tyrosine kinase cell surface growth factor receptor that is expressed in normal epithelial cells. Her2 has an extracellular domain that interacts with extracellular growth factors and an internal tyrosine kinase portion that transmits the external growth signal transduction pathways leading to cell growth and differentiation. Her2 is overexpressed in a significant proportion of malignancies, such as breast cancer, ovarian cancer, prostate cancer and gastric cancers, and is typically associated with a poor prognosis. It is encoded within the genome by HER2/neu, a known proto-oncogene. HER2 is thought to be an orphan receptor, with none of the EGF family of ligands able to activate it. However, ErbB receptors dimerise on ligand binding, and HER2 is the preferential dimerisation partner of other members of the ErbB family. The HER2 gene is a proto-oncogene located at the long arm of human chromosome 17(17q21-q22). HER2/neu (also known as ErbB-2) stands for “Human Epidermal growth factor Receptor 2” and is a protein giving higher aggressiveness in breast cancers. It is a member of the ErbB protein family, more commonly known as the epidermal growth factor receptor family. HER2/neu has also been designated as CD340 (cluster of differentiation 340) and p185. Approximately 15-20 percent of breast cancers have an amplification of the HER2/neu gene or overexpression of its protein product. Overexpression of this receptor in breast cancer is associated with increased disease recurrence and worse prognosis.

Anaplastic Lymphoma Kinase (ALK) tyrosine kinase receptor is an enzyme that in humans is encoded by the ALK gene. The 2;5 chromosomal translocation is frequently associated with anaplastic large cell lymphomas (ALCLs). The translocation creates a fusion gene consisting of the ALK (anaplastic lymphoma kinase) gene and the nucleophosmin (NPM) gene: the 3′ half of ALK, derived from chromosome 2, is fused to the 5′ portion of NPM from chromosome 5. The product of the NPM-ALK fusion gene is oncogenic. Other possible translocations of the ALK gene, such as the elm4 translocation, are also implicated in cancer.

B-Raf proto-oncogene serine/threonine-protein kinase (B-RAF), also known as V-raf murine sarcoma viral oncogene homolog B1, is a protein that in humans is encoded by the BRAF gene. The B-RAF protein is involved in sending signals in cells and in cell growth. The BRAF gene may be mutated, and the B-RAF protein altered, as an inherited mutation which causes birth defects, or as an acquired mutation (oncogene) in adults which causes cancer. Acquired mutations in this gene have also been found in cancers, including non-Hodgkin lymphoma, colorectal cancer, malignant melanoma, papillary thyroid carcinoma, non-small cell lung carcinoma, and adenocarcinoma of lung. More than 30 mutations of the BRAF gene associated with human cancers have been identified. The frequency of BRAF mutations varies widely in human cancers from more than 80% in melanomas, to as little as 0-18% in other tumors, such as 1-3% in lung cancers and 5% in colorectal cancer. In 90% of the cases, a Glu for Val substitution at residue 599 (now referred to as V600E) in the activation segment has been found in human cancers. This mutation has been widely observed in papillary thyroid carcinoma, colorectal cancer and melanomas. Depending on the type of mutation the kinase activity towards MEK may also vary. It has been reported that most of the mutants stimulate enhanced B-RAF kinase activity toward MEK. However, a few mutants act through a different mechanism because although their activity toward MEK is reduced, they adopt a conformation that activates wild-type C-RAF, which then signals to ERK.

KRAS is a protein which in humans is encoded by the KRAS gene. Like other members of the Ras family, the KRAS protein is a GTPase and is an early player in many signal transduction pathways. KRAS is usually tethered to cell membranes because of the presence of an isoprenyl group on its C-terminus. When mutated, KRAS is an oncogene. The protein product of the normal KRAS gene performs an essential function in normal tissue signaling, and the mutation of a KRAS gene is an essential step in the development of many cancers. KRAS acts as a molecular on/off switch, and once it is turned on it recruits and activates proteins necessary for the propagation of growth factor and other receptors' signal, such as c-Raf and PI 3-kinase.

Phosphoinositide 3-kinases (PI 3-kinases or PI3Ks) are a family of enzymes involved in cellular functions such as cell growth, proliferation, differentiation, motility, survival and intracellular trafficking, which in turn are involved in cancer. PI3Ks are a family of related intracellular signal transducer enzymes capable of phosphorylating the 3 position hydroxyl group of the inositol ring of phosphatidylinositol (PtdIns). They are also known as phosphatidylinositol-3-kinases. The pathway, with oncogene PIK3CA and tumor suppressor PTEN (gene) is implicated in insensitivity of cancer tumors to insulin and IGF1, in calorie restriction. PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110α is mutated in many cancers. Many of these mutations cause the kinase to be more active. The PtdIns(3,4,5)P₃ phosphatase PTEN that antagonises PI 3-kinase signaling is absent from many tumors. Hence, PI 3-kinase activity contributes significantly to cellular transformation and the development of cancer.

AKT protein family, which members are also called protein kinases B (PKB) plays an important role in mammalian cellular signaling. Akt kinase is a serine/threonine kinase which is a downstream effector molecule of phosphoinositide 3-kinase and is involved in protecting a cell from apoptosis. Akt kinase is thought to be involved in the progression of cancer because it stimulates cell proliferation and suppresses apoptosis. Akt1 is involved in cellular survival pathways, by inhibiting apoptotic processes. Akt1 is also able to induce protein synthesis pathways, and is therefore a key signaling protein in the cellular pathways that lead to skeletal muscle hypertrophy, and general tissue growth. Since it can block apoptosis, and thereby promote cell survival, Akt1 has been implicated as a major factor in many types of cancer. Akt is known to play a role in the cell cycle. Under various circumstances, activation of Akt was shown to overcome cell cycle arrest in G1 and G2 phases. Moreover, activated Akt may enable proliferation and survival of cells that have sustained a potentially mutagenic impact and, therefore, may contribute to acquisition of mutations in other genes.

Cdk4/cyclin D complexes are involved in phosphorylation of the retinoblastoma protein, which is an essential step in progression of a cell through the G1 phase of the cell cycle. Disruption of Hsp90 activity has been shown to decrease the half life of newly synthesized Cdk4.

Raf-1 is a MAP 3-kinase (MAP3K) which, when activated, can phosphorylate and activate the serine/threonine specific protein kinases ERK1 and ERK2. Activated ERKs play an important role in the control of gene expression involved in the cell division cycle, apoptosis, cell differentiation and cell migration.

The transforming protein of the Rous sarcoma virus, v-src, is a prototype of an oncogene family that induces cellular transformation (i.e., tumorogenesis) by non-regulated kinase activity. Hsp90 has been shown to complex with v-scr and inhibit its degradation.

Hsp90 is required to maintain steroid hormone receptors in conformations capable of binding hormones with high affinity. Inhibition of the action of Hsp90 therefore is expected to be useful in treating hormone-associated malignancies such as breast cancer.

p53 is a tumor suppressor protein that causes cell cycle arrest and apoptosis. Mutation of the p53 gene is found in about half of all human cancers, making it one of the most common genetic alterations found in cancerous cells. In addition, the p53 mutation is associated with a poor prognosis. Wild-type p53 has been shown to interact with Hsp90, but mutated p53 forms a more stable association with Hsp90 than wild-type p53 as a result of its misfolded conformation. A stronger interaction with Hsp90 protects the mutated protein from normal proteolytic degradation and prolongs its half-life. In a cell that is heterozygous for mutated and wild-type p53, inhibition of the stabilizing effect of Hsp90 causes mutant p53 to be degraded and restores the normal transcriptional activity of wild-type p53.

There are two classes of protein kinases (PKs): protein tyrosine kinases (PTKs), which catalyze the phosphorylation of tyrosine kinase residues, and the serine-threonine kinases (STKs), which catalyze the phosphorylation of serine or threonine residues. Growth factor receptors with PTK activity are known as receptor tyrosine kinases. Receptor tyrosine kinases are a family of tightly regulated enzymes, and the aberrant activation of various members of the family is one of the hallmarks of cancer. The receptor tyrosine kinase family can be divided into subgroups that have similar structural organization and sequence similarity within the kinase domain.

The members of the type III group of receptor tyrosine kinases include platelet-derived growth factor receptors (PDGF receptors alpha and beta), colony-stimulating factor receptor (CSF-1R, c-Fms), Fms-like tyrosine kinase (FLT3), and stem cell factor receptor (c-Kit). FLT3 is primarily expressed on immature hematopoietic progenitors and regulates their proliferation and survival.

The FLT3-ITD mutation is also present in about 3% of cases of adult myelodysplastic syndrome and some cases of acute lymphocytic leukemia (ALL). Advani, Current Pharmaceutical Design (2005), 11:3449-3457. FLT3 has been shown to be a client protein of Hsp90, and 17AAG, a benzoquinone ansamycin antibiotic that inhibits Hsp90 activity, has been shown to disrupt the association of FLT3 with Hsp90. The growth of leukemia cells that express either wild type FLT3 or FLT3-ITD mutations was found to be inhibited by treatment with 17AAG. Yao, et al., Clinical Cancer Research (2003), 9:4483-4493.

c-Kit is a membrane type III receptor protein tyrosine kinase which binds Stem Cell Factor (SCF) to its extraellular domain. c-Kit has tyrosine kinase activity and is required for normal hematopoiesis. However, mutations in c-Kit can result in ligand-independent tyrosine kinase activity, autophosphorylation and uncontrolled cell proliferation. Aberrant expression and/or activation of c-Kit have been implicated in a variety of pathologic states. For example, there is evidence of a contribution of c-Kit to neoplastic pathology, including its association with leukemias and mast cell tumors, small cell lung cancer, testicular cancer and some cancers of the gastrointestinal tract and central nervous system. In addition, c-Kit has been implicated in carcinogenesis of the female genital tract, sarcomas of neuroectodermal origin, and Schwann cell neoplasia associated with neurofibromatosis. Yang et al., J Clin Invest. (2003), 112:1851-1861; Viskochil, J Clin Invest. (2003), 112:1791-1793. c-Kit has been shown to be a client protein of Hsp90, and Hsp90 inhibitor 17AAG has been shown to induce apoptosis in Kasumi-1 cells, an acute myeloid leukemia cell line that harbors a mutation in c-Kit.

c-Met is a receptor tyrosine kinase that is encoded by the Met protooncogene and transduces the biological effects of hepatocyte growth factor (HGF), which is also referred to as scatter factor (SF). Jiang, et al., Crit. Rev. Oncol. Hemtol. (1999), 29: 209-248. c-Met and HGF are expressed in numerous tissues, although their expression is normally predominantly confined to cells of epithelial and mesenchymal origin, respectively. c-Met and HGF are required for normal mammalian development and have been shown to be important in cell migration, cell proliferation, cell survival, morphogenic differentiation and the organization of 3-dimensional tubular structures (e.g., renal tubular cells, gland formation, etc.). The c-Met receptor has been shown to be expressed in a number of human cancers. c-Met and its ligand, HGF, have also been shown to be co-expressed at elevated levels in a variety of human cancers, particularly sarcomas. However, because the receptor and ligand are usually expressed by different cell types, c-Met signaling is most commonly regulated by tumor-stroma (tumor-host) interactions. Furthermore, c-Met gene amplification, mutation and rearrangement have been observed in a subset of human cancers. Families with germine mutations that activate c-Met kinase are prone to multiple kidney tumors, as well as tumors in other tissues. Numerous studies have correlated the expression of c-Met and/or HGF/SF with the state of disease progression of different types of cancer, including lung, colon, breast, prostate, liver, pancreas, brain, kidney, ovarian, stomach, skin and bone cancers. Furthermore, the overexpression of c-Met or HGF have been shown to correlate with poor prognosis and disease outcome in a number of major human cancers including lung, liver, gastric and breast.

BCR-ABL is an oncoprotein with tyrosine kinase activity that has been associated with chronic myelogenous leukemia (CML), acute lymphocytic leukemia (ALL) in a subset of patients and acute myelogenous leukemia (AML) in a subset of patients. In fact, the BCR-ABL oncogene has been found in at least 90-95% of patients with CML, about 20% of adults with ALL, about 5% of children with ALL and in about 2% of adults with AML. The BCR-ABL oncoprotein is generated by the translocation of gene sequences from the c-ABL protein tyrosine kinase on chromosome 9 into the BCR sequences on chromosome 22, producing the Philadelphia chromosome. The BCR-ABL gene has been shown to produce at least three alternative chimeric proteins, p230 BCR-ABL, p210 BCR-ABL and p190 BCR-ABL, which have unregulated tyrosine kinase activity. The p210 BCR-ABL fusion protein is most often associated with CML, while the p190 BCR-ABL fusion protein is most often associated with ALL. BCR-ABL has also been associated with a variety of additional hematological malignancies including granulocytic hyperplasia, myelomonocytic leukemia, lymphomas and erythroid leukemia. BCR-ABL fusion proteins exist as complexes with Hsp90 and are rapidly degraded when the action of Hsp90 is inhibited. It has been shown that geldanamycin, a benzoquinone ansamycin antibiotic that disrupts the association of BCR-ABL with Hsp90, results in proteasomal degradation of BCR-ABL and induces apoptosis in BCR-ABL leukemia cells.

Epidermal Growth Factor Receptor (EGFR) is a member of the type 1 subgroup of receptor tyrosine kinase family of growth factor receptors which play critical roles in cellular growth, differentiation and survival. Activation of these receptors typically occurs via specific ligand binding which results in hetero- or homodimerization between receptor family members, with subsequent autophosphorylation of the tyrosine kinase domain. Specific ligands which bind to EGFR include epidermal growth factor (EGF), transforming growth factor α (TGFα), amphiregulin and some viral growth factors. Activation of EGFR triggers a cascade of intracellular signaling pathways involved in both cellular proliferation (the ras/raf/MAP kinase pathway) and survival (the PI3 kinase/Akt pathway). Members of this family, including EGFR and HER2, have been directly implicated in cellular transformation.

A number of human malignancies are associated with aberrant or overexpression of EGFR and/or overexpression of its specific ligands. Gullick, Br. Med. Bull. (1991), 47:87-98; Modijtahedi & Dean, Int. J. Oncol. (1994), 4:277-96; Salomon, et al., Crit. Rev. Oncol. Hematol. (1995), 19:183-232. Aberrant or overexpression of EGFR has been associated with an adverse prognosis in a number of human cancers, including head and neck, breast, colon, prostate, lung (e.g., NSCLC, adenocarcinoma and squamous lung cancer), ovarian, gastrointestinal cancers (gastric, colon, pancreatic), renal cell cancer, bladder cancer, glioma, gynecological carcinomas and prostate cancer. In some instances, overexpression of tumor EGFR has been correlated with both chemoresistance and a poor prognosis. Lei, et al., Anti-cancer Res. (1999), 19:221-28; Veale, et al., Br. J. Cancer (1993); 68:162-65. Mutations in EGFR are associated with many types of cancer as well. For example, EGFR mutations are highly prevalent in non-mucinous BAC patients. Finberg, et al., J. Mol. Diagnostics (2007) 9(3):320-26.

Hsp90 has been shown by mutational analysis to be necessary for the survival of normal eukaryotic cells. However, Hsp90 is over expressed in many tumor types indicating that it may play a significant role in the survival of cancer cells, and that cancer cells may be more sensitive to inhibition of Hsp90 than normal cells. For example, cancer cells typically have a large number of mutated and overexpressed oncoproteins that are dependent on Hsp90 for folding. In addition, because the environment of a tumor is typically hostile due to hypoxia, nutrient deprivation, acidosis, etc., tumor cells may be especially dependent on Hsp90 for survival. Moreover, inhibition of Hsp90 causes the simultaneous inhibition of a number of oncoproteins, hormone receptors and transcription factors, thus making it an attractive target for an anti-cancer agent. In fact, benzoquinone ansamycins, a family of natural products that inhibit Hsp90, have shown evidence of therapeutic activity in clinical trials.

Although initially promising, first generation Hsp90 inhibitors, the benzoquinone ansamycins, and their derivatives, suffer from some limitations. For example, they have low oral bioavailability and their limited solubility makes them difficult to formulate. In addition, they are metabolized by polymorphic cytochrome P450 CYP3A4 and are a substrate for the P-glycoprotein export pump involved in the development of multidrug resistance. Additionally, the ansamycin class of Hsp90 inhibitors has shown serious toxicity problems. Therefore, a need exists for new therapeutics that improve the prognosis of cancer patients and that reduce or overcome the limitations of currently used anti-cancer agents.

Despite the availability of multiple therapeutic regimens to treat proliferative disorders such as cancer, many patients do not respond to any treatments. Of those that do respond to standard therapies, the effect is usually short-lived as resistance develops to the initial therapeutic regimens. There is an immediate need in the art for improvement in cancer therapies, both in terms of the proportion of patients who respond to therapy and the survival benefit imparted.

### SUMMARY OF THE INVENTION

It has been found that certain specific treatment regimens with ganetespib are particularly effective in treating certain types of cancer while maintaining favorable safety profile of ganetespib. In an embodiment, the method includes treating, managing, or ameliorating cancer, or one or more symptoms thereof, comprising administering to a subject in need thereof an effective amount of ganetespib, or a pharmaceutically acceptable salt thereof. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with a taxane derivative. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with a taxane derivative.

In another embodiment, methods include treating, managing, or ameliorating p53-mutated cancer, or one or more symptoms thereof, by administering to a subject in need thereof an effective amount of ganetespib, or a pharmaceutically acceptable salt thereof, in combination with a taxane derivative. In another embodiment, methods include treating, managing, or ameliorating p53-mutated solid tumor, non-small cell lung cancer, breast cancer, ovarian cancer, prostate cancer, pancreatic cancer, inflammatory breast cancer, peritoneal cancer, bladder cancer, or colon cancer, or one or more symptoms thereof, by administering to a subject in need thereof an effective amount of ganetespib, or a pharmaceutically acceptable salt thereof, in combination with a taxane derivative. In an embodiment, the ovarian cancer is metastatic. In an embodiment, the ovarian cancer is platinum-resistant. In yet another embodiment, the ovarian cancer is metastatic and platinum-resistant. In an embodiment, the breast cancer is triple negative breast cancer (TNBC). Yet another embodiment includes identifying and/or evaluating patients having p53-mutated cancers (e.g., metastatic and/or platinum-resistant cancers; ovarian, breast or triple-negative breast cancer (TNBC); or metastatic and/or platinum-resistant ovarian, breast or triple-negative breast cancer); and so identified such patients as being candidates for treatment, treating them in accordance with methods described herein. In an embodiment, the method includes treating, managing, or ameliorating cancer, or one or more symptoms thereof, comprising administering to a subject in need thereof an effective amount of ganetespib, or a pharmaceutically acceptable salt thereof, in combination with docetaxel or paclitaxel. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with docetaxel or paclitaxel.

In another embodiment, methods include treating, managing, or ameliorating p53-mutated cancer, or one or more symptoms thereof, by administering to a subject in need thereof an effective amount of ganetespib, or a pharmaceutically acceptable salt thereof, in combination with a taxane derivative. In another embodiment, methods include treating, managing, or ameliorating p53-mutated solid tumor, non-small cell lung cancer, breast cancer, ovarian cancer, prostate cancer, pancreatic cancer, inflammatory breast cancer, peritoneal cancer, bladder cancer, or colon cancer, or one or more symptoms thereof, by administering to a subject in need thereof an effective amount of ganetespib, or a pharmaceutically acceptable salt thereof, in combination with a taxane derivative. In an embodiment, the ovarian cancer is metastatic. In an embodiment, the ovarian cancer is platinum-resistant. In yet another embodiment, the ovarian cancer is metastatic and platinum-resistant. In an embodiment, the breast cancer is triple negative breast cancer (TNBC). In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of a taxane derivative, and an effective amount of a platinum-containing anti-cancer agent. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of a taxane derivative, and an effective amount of a platinum-containing anti-cancer agent. In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of docetaxel or paclitaxel, and an effective amount of cisplatin or carboplatin. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of docetaxel or paclitaxel, and an effective amount of cisplatin or carboplatin.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of a platinum-containing anti-cancer agent, and an effective amount of an antimetabolite. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of a platinum-containing anti-cancer agent, and an effective amount of an antimetabolite.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of cisplatin, and an effective amount of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of cisplatin, and an effective amount of pemetrexed.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of carboplatin, and an effective amount of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of carboplatin, and an effective amount of pemetrexed.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of a taxane derivative, an effective amount of an anthracycline derivative, and an effective amount of an alkylating agent. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with a taxane derivative, an effective amount of an anthracycline derivative, and an effective amount of an alkylating agent.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of docetaxel or paclitaxel, an effective amount of doxorubicin, and an effective amount of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with docetaxel or paclitaxel, an effective amount of doxorubicin, and an effective amount of cyclophosphamide.

### DETAILED DESCRIPTION OF THE INVENTION

### Definitions

Unless otherwise specified, the below terms used herein are defined as follows:

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “Hsp90” includes each member of the family of heat shock proteins having a mass of about 90-kiloDaltons. For example, in humans the highly conserved Hsp90 family includes the cytosolic Hsp90α and Hsp9013 isoforms, as well as GRP94, which is found in the endoplasmic reticulum, and HSP75/TRAP1, which is found in the mitochondrial matrix.

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which said therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

The taxanes are anti-cancer agents that include paclitaxel (Taxol®) and docetaxel (Taxotere®). Both drugs have proved to be effective in the treatment of a variety of solid tumors including breast, ovarian, lung, and bladder cancers. Thus, the term “paclitaxel analog” is defined herein to mean a compound which has the basic paclitaxel skeleton and which stabilizes microtubule formation. Many analogs of paclitaxel are known, including docetaxel. In addition, a paclitaxel analog can also be bonded to or be pendent from a pharmaceutically acceptable polymer, such as a polyacrylamide. The term “paclitaxel analog”, as it is used herein, includes such polymer linked taxanes.

Anthracyclines as used herein are a class of drugs used in cancer therapy. These compounds are used to treat many cancers including leukemia, lymphomas, breast, uterine, ovarian and lung cancers. Typical anthracyclines include daunorubicin, doxorubicin, epirubucin, idarubicin, and valrubucin.

A platinum-containing anticancer agent as used herein includes cisplatin, carboplatin, oxaliplatin, satraplatin, picoplatin, nedaplatin, and triplatin.

An antimetabolite as used herein includes methotrexate, pemetrexed, cytarabine (also called Ara-C), nelarabine (also called Ara-G), 5-fluorouracil, capecitabine or their derivatives.

An alkylating anticancer agent, or an alkylating antineoplastic agent as used herein includes cyclophosphamide, mechlorethamine, uramustine, melphalan, chlorambucil, ifosfamide, bendamustine, carmustine, lomustine, streptozocin, and busulfan.

The term “vascular endothelial growth factor inhibitor” or “VEGF inhibitor” includes any compounds that disrupt the function of vascular endothelial growth factor A (VEGF) production within a cell. VEGF inhibitors are another class of anticancer agents. VEGF inhibitors include drugs such as bevacizumab (Avastin®), sunitinib (Sutent®), and sorafenib (Nexavar®). Examples of VEGF receptor inhibitors include sunitinib and sorafenib. Monoclonal antibody therapies, such as bevacizumab, that block VEGF are described in U.S. Pat. Nos. 6,884,879, 7,060,269, and 7,297,334.

The dosages of other anti-cancer agents, which have been or are currently being used to prevent, treat, manage, or ameliorate disorders, such cancer, or one or more symptoms thereof can be used in the combination therapies of the invention. Preferably, dosages lower than those which have been or are currently being used to prevent, treat, manage, or ameliorate cancer, or one or more symptoms thereof, are used in the combination therapies of the invention. The recommended dosages of agents currently used for the prevention, treatment, management, or amelioration of cancer, or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Some of the disclosed pharmaceutical compositions can be particularly effective at treating subjects with proliferative disorders. In one embodiment, the proliferative disorder is cancer. In one embodiment, the pharmaceutical composition is administered to a subject whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

Other anti-proliferative or anti-cancer therapies may be combined with the compounds described herein to treat proliferative diseases and cancer. Other therapies or anti-cancer agents that may be used in combination with the inventive anti-cancer agents described herein include surgery, radiotherapy (including, but not limited to, gamma-radiation, neutron beam radiotherapy, electron beam radiotherapy, proton therapy, brachytherapy, and systemic radioactive isotopes), endocrine therapy, biologic response modifiers (including, but not limited to, interferons, interleukins, and tumor necrosis factor (TNF)), hyperthermia and cryotherapy, agents to attenuate any adverse effects (e.g., antiemetics), and other approved chemotherapeutic drugs.

A pharmaceutically acceptable carrier may contain inert ingredients which do not unduly inhibit the biological activity of the compound(s). The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

An “effective amount” is that amount sufficient to treat a disease in a subject. A therapeutically effective amount can be administered in one or more administrations.

The term “effective amount” includes an amount of ganetespib which is sufficient to treat the cancer, to reduce or ameliorate the severity, duration, or progression of cancer, to retard or halt the advancement of cancer, to cause the regression of cancer, to delay the recurrence, development, onset, or progression of a symptom associated with cancer, or to enhance or improve the therapeutic effect(s) of another therapy. For example, an effective amount can induce, for example, a complete response, a partial response, or stable disease; as determined, for example, using RESIST criteria.

An “effective amount” of a therapeutic agent produces a desired response. Having a positive response to treatment with a therapeutic agent is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). It is understood that an “effective amount” need not be curative.

An effective amount of ganetespib is understood as an amount of ganetespib to improves outcome relative to an appropriate control group, e.g., an untreated group, a group treated with a combination of therapies not including ganetespib. Methods to select appropriate control groups and to perform comparative analyses are within the ability of those of skill in the art.

The precise amount of compound administered to provide an “effective amount” of ganetespib to the subject will depend on the mode of administration, the type and severity of the cancer and on the characteristics of the subject, such as general health, age, sex, body weight and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th ed., 2003).

The dosage of an individual agent used in combination therapy may be equal to or lower than the dose of an individual therapeutic agent when given independently to treat, manage, or ameliorate a disease or disorder, or one or more symptoms thereof. In one embodiment, the disease or disorder being treated with a combination therapy is a triple-negative breast cancer.

In an embodiment, the amount of ganetespib administered is from about 2 mg/m² to about 500 mg/m², for example, from about 100 mg/m² to about 500 mg/m², from about 125 mg/m² to about 500 mg/m², from about 150 mg/m² to about 500 mg/m² or from about 175 mg/m² to about 500 mg/m². In an embodiment, the amount of ganetespib administered is about 100 mg/m² to about 300 mg/m², from about 125 mg/m² to about 300 mg/m², from about 150 mg/m² to about 300 mg/m² or from about 175 mg/m² to about 300 mg/m². In some embodiments, the amount of ganetespib administered is about 2 mg/m², 4 mg/m², about 7 mg/m², about 10 mg/m², about 14 mg/m², about 19 mg/m², about 23 mg/m², about 25 mg/m², about 33 mg/m², about 35 mg/m², about 40 mg/m², about 48 mg/m², about 49 mg/m², about 50 mg/m², about 65 mg/m², about 75 mg/m², about 86 mg/m², about 100 mg/m², about 110 mg/m², about 114 mg/m², about 120 mg/m², about 144 mg/m², about 150 mg/m², about 173 mg/m², about 180 mg/m², about 200 mg/m², about 216 mg/m² or about 259 mg/m².

In one embodiment, the pharmaceutical composition is formulated to deliver a dose of about 50 mg/m², about 75 mg/m², about 100 mg/m², about 125 mg/m², about 150 mg/m², about 175 mg/m², about 200 mg/m², about 225 mg/m², or about 250 mg/m². Administration of a compound described herein may occur once a week or twice a week. In one embodiment, the pharmaceutical composition is formulated at a dose of about 200 mg/m² and administered once a week.

In one embodiment, the pharmaceutical composition is administered parentally. In one embodiment, the pharmaceutical composition is administered intravenously through an in-dwelling port or through peripheral access. In one embodiment, the pharmaceutical composition is administered through a silicone catheter in an in-dwelling port.

In one embodiment, the pharmaceutical compositions described herein are administered once or twice every week for three out of four weeks, with the fourth week being a “rest week” for the subject being treated. In one embodiment, the pharmaceutical compositions described herein can also be administered once or twice a week for more than three consecutive weeks, with no rest week.

The language “twice-weekly” includes administration of ganetespib two times in about 7 days. For example, the first dose of ganetespib is administered on day 1, and the second dose of ganetespib may be administered on day 2, day 3, day 4, day 5, day 6 or day 7. In some embodiments, the twice-weekly administration occurs on days 1 and 3 or days 1 and 4.

In some embodiments, ganetespib is cyclically administered twice-weekly. For example, ganetespib is administered for a first period of time, followed by a “dose-free” period, then administered for a second period of time. The language “dose-free” includes the period of time in between the first dosing period and the second dosing period in which no ganetespib is administered to the subject. A preferred cycle is administering ganetespib at a dose described above two times during the week for three consecutive weeks followed by one dose-free week. This cycle is then repeated, as described below.

The language “one cycle” includes the first period of time during which ganetespib is administered, followed by a dose-free period of time. The dosing cycle can be repeated and one of skill in the art will be able to determine the appropriate length of time for such a cyclical dosing regimen. In an embodiment, the cycle is repeated at least once. In an embodiment, the cycle is repeated two or more times. In an embodiment, the cycle is repeated 3, 4, 5, 6, 7, 8, 9, 10, 11, 12, 13, 14, 15 or more times, or as many times as medically necessary as determined by one of skill in the art, e.g., as long as the subject exhibits a response with no dose limiting toxicities. In an embodiment, the cycle is repeated until the patient has been determined to be in partial remission (e.g., 50% or greater reduction in the measurable parameters of tumor growth) or complete remission (e.g., absence of cancer). One of skill in the art can determine a patient's remission status using routine methods well known in the art.

As used herein, the term “in combination” refers to the use of more than one therapeutic agent (e.g., 2, 3, 4, 5, 6, 7, 8, 9, 10, or more). The use of the term “in combination” does not restrict the order in which the therapeutic agents are administered to a subject afflicted with cancer. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent or treatment, such as an anti-cancer agent, to a subject with cancer. In certain embodiments, one agent may be administered more frequently than the other agent such that multiple doses of one agent are administered for each dose of the other agent(s).

In an embodiment, the method includes treating, managing, or ameliorating cancer, or one or more symptoms thereof, comprising administering to a subject in need thereof an effective amount of ganetespib, or a pharmaceutically acceptable salt thereof. In an embodiment, the method includes treating, managing, or ameliorating solid tumor, non-small cell lung cancer, breast cancer, ovarian cancer, fallopian tube cancer, or peritoneal cancer, or one or more symptoms thereof, comprising administering to a subject in need thereof an effective amount of ganetespib, or a pharmaceutically acceptable salt thereof.

In an embodiment, the method includes treating, managing, or ameliorating cancer, or one or more symptoms thereof, comprising administering to a subject in need thereof about 75 mg/m², or about 100 mg/m², or about 1²5 mg/m², or about 150 mg/m², or about 175 mg/m², or about 200 mg/m², or about 225 mg/m² of ganetespib, or a pharmaceutically acceptable salt thereof. In an embodiment, the method includes treating, managing, or ameliorating solid tumor, non-small cell lung cancer, breast cancer, ovarian cancer, fallopian tube cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with a taxane derivative. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with a taxane derivative. In another embodiment, methods include treating, managing, or ameliorating p53-mutated cancer, or one or more symptoms thereof, by administering to a subject in need thereof an effective amount of ganetespib, or a pharmaceutically acceptable salt thereof, in combination with a taxane derivative. In another embodiment, methods include treating, managing, or ameliorating p53-mutated solid tumor, non-small cell lung cancer, breast cancer, ovarian cancer, prostate cancer, pancreatic cancer, inflammatory breast cancer, peritoneal cancer, bladder cancer, or colon cancer, or one or more symptoms thereof, by administering to a subject in need thereof an effective amount of ganetespib, or a pharmaceutically acceptable salt thereof, in combination with a taxane derivative. In an embodiment, the ovarian cancer is metastatic. In an embodiment, the ovarian cancer is platinum-resistant. In yet another embodiment, the ovarian cancer is metastatic and platinum-resistant. In an embodiment, the breast cancer is triple negative breast cancer (TNBC).

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a taxane derivative. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a taxane derivative.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of docetaxel or paclitaxel. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of docetaxel or paclitaxel. In another embodiment, methods include treating, managing, or ameliorating p53-mutated cancer, or one or more symptoms thereof, by administering to a subject in need thereof an effective amount of ganetespib, or a pharmaceutically acceptable salt thereof, in combination with a taxane derivative. In another embodiment, methods include treating, managing, or ameliorating p53-mutated solid tumor, non-small cell lung cancer, breast cancer, ovarian cancer, prostate cancer, pancreatic cancer, inflammatory breast cancer, peritoneal cancer, bladder cancer, or colon cancer, or one or more symptoms thereof, by administering to a subject in need thereof an effective amount of ganetespib, or a pharmaceutically acceptable salt thereof, in combination with a taxane derivative. In an embodiment, the ovarian cancer is metastatic. In an embodiment, the ovarian cancer is platinum-resistant. In yet another embodiment, the ovarian cancer is metastatic and platinum-resistant. In an embodiment, the breast cancer is triple negative breast cancer (TNBC).

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered about 1 hour following the completion of ganetespib infusion. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered about 1 hour following the completion of ganetespib infusion.

In an embodiment, the method includes treating, managing, or ameliorating cancer, or one or more symptoms thereof, comprising administering to a subject in need thereof about 75 mg/m2, or about 100 mg/m2, or about 125 mg/m2, or about 150 mg/m2, or about 175 mg/m2, or about 200 mg/m2, or about 225 mg/m2 of ganetespib, or a pharmaceutically acceptable salt thereof, in combination with about 60 mg/m2 of docetaxel, wherein docetaxel is administered about 1 hour following the completion of ganetespib infusion. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m2, or about 100 mg/m2, or about 125 mg/m2, or about 150 mg/m2, or about 175 mg/m2, or about 200 mg/m², or about 225 mg/m² of ganetespib, or a pharmaceutically acceptable salt thereof, in combination with about 60 mg/m² of docetaxel, wherein docetaxel is administered about 1 hour following the completion of ganetespib infusion.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered on day 1 following the administration of ganetespib, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered on day 1 following the administration of ganetespib, and wherein a second dose of an effective amount of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 60 mg/m² of docetaxel, wherein docetaxel is administered on day 1 following the administration of ganetespib, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 60 mg/m² of docetaxel, wherein docetaxel is administered on day 1 following the administration of ganetespib, and wherein a second dose of an effective amount of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered on day 1 following the administration of ganetespib, and wherein a second dose of about 75 mg/m² of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered on day 1 following the administration of ganetespib, and wherein a second dose of about 75 mg/m² of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 60 mg/m² of docetaxel, wherein docetaxel is administered on day 1 following the administration of ganetespib, and wherein a second dose of 75 mg/m² of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 60 mg/m² of docetaxel, wherein docetaxel is administered on day 1 following the administration of ganetespib, and wherein a second dose of about 75 mg/m² of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered on day 1 following the administration of ganetespib, and wherein a second dose of about 150 mg/m² of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, wherein docetaxel is administered on day 1 following the administration of ganetespib, and wherein a second dose of about 150 mg/m² of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 60 mg/m² of docetaxel, wherein docetaxel is administered on day 1 following the administration of ganetespib, and wherein a second dose of 150 mg/m² of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 60 mg/m² of docetaxel, wherein docetaxel is administered on day 1 following the administration of ganetespib, and wherein a second dose of about 150 mg/m² of ganetespib is administered on day 15.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of a taxane derivative, and an effective amount of a platinum-containing anti-cancer agent. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of a taxane derivative, and an effective amount of a platinum-containing anti-cancer agent.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a taxane derivative, and an effective amount of a platinum-containing anti-cancer agent. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a taxane derivative, and an effective amount of a platinum-containing anti-cancer agent.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of paclitaxel, and an effective amount of carboplatin. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of paclitaxel, and an effective amount of carboplatin.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, wherein a second dose of an effective amount of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, and wherein a second dose of about 75 mg/m² of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m2, or about 100 mg/m2, or about 1²5 mg/m2, or about 150 mg/m2, or about 175 mg/m2, or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, and wherein a second dose of about 75 mg/m² of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, and wherein a second dose of about 100 mg/m² of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, and wherein a second dose of about 100 mg/m² of ganetespib is administered on day 15.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, and wherein a second dose of about 150 mg/m² of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 200 mg/m² of paclitaxel, and about AUC 6 of carboplatin, wherein both paclitaxel and carboplatin are administered on day 1 following the administration of ganetespib, and wherein a second dose of about 150 mg/m² of ganetespib is administered on day 15.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of a platinum-containing anti-cancer agent, and an effective amount of an antimetabolite. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of a platinum-containing anti-cancer agent, and an effective amount of an antimetabolite.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a platinum-containing anti-cancer agent, and an effective amount of an antimetabolite. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a platinum-containing anti-cancer agent, and an effective amount of an antimetabolite.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of cisplatin, and an effective amount of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of cisplatin, and an effective amount of pemetrexed.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of cisplatin, and an effective amount of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of cisplatin, and an effective amount of pemetrexed.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with about 75 mg/m² of cisplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with about 75 mg/m² of cisplatin, and about 500 mg/m² of pemetrexed.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of cisplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of cisplatin, and about 500 mg/m² of pemetrexed.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about 75 mg/m² of cisplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about 75 mg/m² of cisplatin, and about 500 mg/m² of pemetrexed.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about 75 mg/m² of cisplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about 75 mg/m² of cisplatin, and about 500 mg/m² of pemetrexed.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about 75 mg/m² of cisplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about 75 mg/m² of cisplatin, and about 500 mg/m² of pemetrexed.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following ganetespib administration. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following ganetespib administration.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following ganetespib administration. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following ganetespib administration.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following ganetespib administration. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following ganetespib administration.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following the first ganetespib administration, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following the first ganetespib administration, and wherein a second dose of an effective amount of ganetespib is administered on day 15.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following the first ganetespib administration, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following the first ganetespib administration, and wherein a second dose of an effective amount of ganetespib is administered on day 15.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following the first ganetespib administration, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about 75 mg/m² of cisplatin, and about 500 mg/m² of pemetrexed, wherein both cisplatin and pemetrexed are administered on day 1 following the first ganetespib administration, and wherein a second dose of an effective amount of ganetespib is administered on day 15.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of carboplatin, and an effective amount of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with an effective amount of carboplatin, and an effective amount of pemetrexed.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of carboplatin, and an effective amount of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of carboplatin, and an effective amount of pemetrexed.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with about AUC 6 of carboplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with about AUC 6 of carboplatin, and about 500 mg/m² of pemetrexed.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about AUC 6 of carboplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about AUC 6 of carboplatin, and about 500 mg/m² of pemetrexed.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about AUC 6 of carboplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about AUC 6 of carboplatin, and about 500 mg/m² of pemetrexed.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about AUC 6 of carboplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about AUC 6 of carboplatin, and about 500 mg/m² of pemetrexed.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about AUC 6 of carboplatin, and about 500 mg/m² of pemetrexed. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about AUC 6 of carboplatin, and about 500 mg/m² of pemetrexed.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following ganetespib administration. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following ganetespib administration.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following ganetespib administration. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following ganetespib administration.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following ganetespib administration. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following ganetespib administration.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following the first ganetespib administration, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about AUC 6 of carboplatin, and about 500 mg/m² of pemetrexed, wherein both carbopaltin and pemetrexed are administered on day 1 following the first ganetespib administration, and wherein a second dose of an effective amount of ganetespib is administered on day 15.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following the first ganetespib administration, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following the first ganetespib administration, and wherein a second dose of an effective amount of ganetespib is administered on day 15.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following the first ganetespib administration, and wherein a second dose of an effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about AUC 6 of carboplatin, and about 500 mg/m² of pemetrexed, wherein both carboplatin and pemetrexed are administered on day 1 following the first ganetespib administration, and wherein a second dose of an effective amount of ganetespib is administered on day 15.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of a taxane derivative, an effective amount of an anthracycline derivative, and an effective amount of an alkylating agent. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with a taxane derivative, an effective amount of an anthracycline derivative, and an effective amount of an alkylating agent.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a taxane derivative, an effective amount of an anthracycline derivative, and an effective amount of an alkylating agent. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of a taxane derivative, an effective amount of an anthracycline derivative, and an effective amount of an alkylating agent.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with an effective amount of docetaxel, an effective amount of doxorubicin, and an effective amount of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with docetaxel, an effective amount of doxorubicin, and an effective amount of cyclophosphamide.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of docetaxel, an effective amount of doxorubicin, and an effective amount of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with an effective amount of docetaxel, an effective amount of doxorubicin, and an effective amount of cyclophosphamide.

In an embodiment, the method includes treating, managing, or ameliorating cancer, or one or more symptoms thereof, comprising administering to a subject in need thereof an effective amount of ganetespib, or a pharmaceutically acceptable salt thereof, in combination with about 75 mg/m² of docetaxel, about 60 mg/m² of doxorubicin, and about 600 mg/m² of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof an effective amount of ganetespib, or a pharmaceutically acceptable salt thereof, in combination with about 75 mg/m² of docetaxel, about 60 mg/m² of doxorubicin, and about 600 mg/m² of cyclophosphamide.

In an embodiment, the method includes treating, managing, or ameliorating cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, about 60 mg/m² of doxorubicin, and about 600 mg/m² of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m², or about 100 mg/m², or about 125 mg/m², or about 150 mg/m², or about 175 mg/m², or about 200 mg/m², or about 225 mg/m² of ganetespib, or a pharmaceutically acceptable salt thereof, in combination with about 75 mg/m² of docetaxel, about 60 mg/m² of doxorubicin, and about 600 mg/m² of cyclophosphamide.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about 75 mg/m² of docetaxel, about 60 mg/m² of doxorubicin, and about 600 mg/m² of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about 75 mg/m² of docetaxel, about 60 mg/m² of doxorubicin, and about 600 mg/m² of cyclophosphamide.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about 75 mg/m² of docetaxel, about 60 mg/m² of doxorubicin, and about 600 mg/m² of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about 75 mg/m² of docetaxel, about 60 mg/m² of doxorubicin, and about 600 mg/m² of cyclophosphamide.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about 75 mg/m² of docetaxel, about 60 mg/m² of doxorubicin, and about 600 mg/m² of cyclophosphamide. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about 75 mg/m² of docetaxel, about 60 mg/m² of doxorubicin, and about 600 mg/m² of cyclophosphamide.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamide are administered on day 1 and docetaxel on day 22 following the administration of ganetespib. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the administration of ganetespib.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the administration of ganetespib. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the administration of ganetespib.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the administration of ganetespib. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the administration of ganetespib.

In an embodiment, the method includes treating, managing, or ameliorating cancer, or one or more symptoms thereof, comprising administering to a subject in need thereof about 75 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the first administration of ganetespib, and wherein a second dose of effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 75 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the first administration of ganetespib, wherein a second dose of effective amount of ganetespib is administered on day 15.

In an embodiment, the method includes treating, managing, or ameliorating cancer, or one or more symptoms thereof, comprising administering to a subject in need thereof about 100 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the first administration of ganetespib, wherein a second dose of effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 100 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the first administration of ganetespib, wherein a second dose of effective amount of ganetespib is administered on day 15.

In an embodiment, the method includes treating, managing, or ameliorating cancer, or one or more symptoms thereof, comprising administering to a subject in need thereof about 150 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the first administration of ganetespib, wherein a second dose of effective amount of ganetespib is administered on day 15. In an embodiment, the method includes treating, managing, or ameliorating solid tumor, non-small cell lung cancer, breast cancer, ovarian cancer, fallopian tube cancer, prostate cancer, pancreatic cancer, inflammatory breast cancer, or peritoneal cancer, or one or more symptoms thereof, comprising administering to a subject in need thereof about 150 mg/m² of ganetespib, or a pharmaceutically acceptable salt thereof, in combination with about 75 mg/m² of docetaxel, about 60 mg/m² of doxorubicin, and about 600 mg/m² of cyclophosphamide, where both doxorubicin and cyclophosphamine are administered on day 1 and docetaxel on day 22 following the first administration of ganetespib, wherein a second dose of effective amount of ganetespib is administered on day 15.

The invention also provides a pharmaceutical composition comprising ganetespib or a pharmaceutically acceptable salt thereof, a taxane derivative, a platinum-containing anti-cancer agent, and/or a pharmaceutically acceptable carrier. The invention also provides a pharmaceutical composition ganetespib or a pharmaceutically acceptable salt thereof, docetaxel or paclitaxel, cisplatin or carboplatin, and/or a pharmaceutically acceptable carrier.

The invention also provides a pharmaceutical composition comprising ganetespib or a pharmaceutically acceptable salt thereof, an antimetabolite anticancer agent, a platinum-containing anti-cancer agent, and/or a pharmaceutically acceptable carrier. The invention also provides a pharmaceutical composition ganetespib or a pharmaceutically acceptable salt thereof, pemetrexed, cisplatin or carboplatin, and/or a pharmaceutically acceptable carrier.

The invention also provides a pharmaceutical composition comprising ganetespib or a pharmaceutically acceptable salt thereof, a taxane derivative, an anthracycline derivative, an anticancer alkylating agent, and/or a pharmaceutically acceptable carrier. The invention also provides a pharmaceutical composition ganetespib or a pharmaceutically acceptable salt thereof, docetaxel or paclitaxel, doxorubicin, cyclophosphamide, and/or a pharmaceutically acceptable carrier.

The invention also provides the use of ganetespib or a pharmaceutically acceptable salt thereof, in combination with a taxane derivative, and a platinum-containing anti-cancer agent for the manufacture of a medicament for the treatment of a subject with a cancer. The invention also provides the use of ganetespib or a pharmaceutically acceptable salt thereof, in combination with a taxane derivative, and a platinum-containing anti-cancer agent for the manufacture of a medicament for the treatment of a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with docetaxel or paclitaxel, and cisplatin or carboplatin for the manufacture of a medicament for the treatment of a subject with a cancer. The invention also provides the use of ganetespib or a pharmaceutically acceptable salt thereof, in combination with docetaxel or paclitaxel, and cisplatin or carboplatin for the manufacture of a medicament for the treatment of a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with a taxane derivative, and an antimetabolite anticancer agent for the manufacture of a medicament for the treatment of a subject with a cancer. The invention also provides the use of ganetespib or a pharmaceutically acceptable salt thereof, in combination with a taxane derivative, and an antimetabolite anti-cancer agent for the manufacture of a medicament for the treatment of a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with docetaxel or paclitaxel, and pemetrexed for the manufacture of a medicament for the treatment of a subject with a cancer. The invention also provides the use of ganetespib or a pharmaceutically acceptable salt thereof, in combination with docetaxel or paclitaxel, and pemetrexed for the manufacture of a medicament for the treatment of a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with a taxane derivative, an anthracline derivative, and an alkylating anti-cancer agent for the manufacture of a medicament for the treatment of a subject with a cancer. The invention also provides the use of ganetespib or a pharmaceutically acceptable salt thereof, in combination with a taxane derivative, an anthracycline derivative, and an alkylating anti-cancer agent for the manufacture of a medicament for the treatment of a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with docetaxel or paclitaxel, doxorubicin, and cyclophosphamide for the manufacture of a medicament for the treatment of a subject with a cancer. The invention also provides the use of ganetespib or a pharmaceutically acceptable salt thereof, in combination with docetaxel or paclitaxel, doxorubicin, and cyclophosphamide for the manufacture of a medicament for the treatment of a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with a taxane derivative, and a platinum-containing anti-cancer agent in treating a subject with a cancer. The invention also provides the use of ganetespib or a pharmaceutically acceptable salt thereof, in combination with a taxane derivative, and a platinum-containing anti-cancer agent in treating a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with docetaxel or paclitaxel, and cisplatin or carboplatin in treating a subject with cancer. The invention also provides the use of ganetespib or a pharmaceutically acceptable salt thereof, in combination with docetaxel or paclitaxel, and cisplatin or carboplatin in treating a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with a taxane derivative, and an antimetabolite anticancer agent in treating a subject with cancer. The invention also provides the use of ganetespib or a pharmaceutically acceptable salt thereof, in combination with a taxane derivative, and an antimetabolite anti-cancer agent in treating a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with docetaxel or paclitaxel, and pemetrexed in treating a subject with cancer. The invention also provides the use of ganetespib or a pharmaceutically acceptable salt thereof, in combination with docetaxel or paclitaxel, and pemetrexed in treating a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with a taxane derivative, an anthracline derivative, and an alkylating anti-cancer agent in treating a subject with a cancer. The invention also provides the use of ganetespib or a pharmaceutically acceptable salt thereof, in combination with a taxane derivative, an anthracycline derivative, and an alkylating anti-cancer agent in treating a subject with solid tumor, non-small cell lung cancer, breast cancer, ovarian cancer, fallopian tube cancer, prostate cancer, pancreatic cancer, inflammatory breast cancer, or peritoneal cancer.

The invention also provides the use of ganetespib or a pharmaceutically acceptable salt thereof, in combination with docetaxel or paclitaxel, doxorubicin, and cyclophosphamide in treating a subject with a cancer. The invention also provides the use of ganetespib or a pharmaceutically acceptable salt thereof, in combination with docetaxel or paclitaxel, doxorubicin, and cyclophosphamide in treating a subject with solid tumor, non-small cell lung cancer, breast cancer, ovarian cancer, fallopian tube cancer, prostate cancer, pancreatic cancer, inflammatory breast cancer, or peritoneal cancer.

Ganetespib and optionally, one or more additional anti-cancer agents, can be administered to a subject by routes known to one of skill in the art. Examples of routes of administration include, but are not limited to, parenteral, e.g., intravenous, intradermal, subcutaneous, oral, intranasal (e.g., inhalation), transdermal, topical, transmucosal, and rectal administration. The agents can be administered by different routes of administration.

Ganetespib, one or more additional anti-cancer agents, may be formulated with a pharmaceutically acceptable carrier, diluent, or excipient as a pharmaceutical composition. Pharmaceutical compositions and dosage forms of the invention comprise one or more active ingredients in relative amounts and formulated in such a way that a given pharmaceutical composition or dosage form can be used to treat cancer. Administration in combination does not require co-formulation.

A pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In some embodiments, ganetespib is formulated at a concentration of 8 mg/mL in 90% v/v PEG 300 and 10% v/v Polysorbate 80 for intravenous administration.

In an embodiment, the invention also provides a pharmaceutical composition further comprises one or more other therapies (e.g., one or more therapeutic agents that are currently being used, have been used, are known to be useful or in development for use in the treatment or amelioration of a proliferative disorder, such as cancer, or one or more symptoms associated with said proliferative disorder). In one embodiment, the pharmaceutical composition further comprises an additional pharmaceutically acceptable co-solvent. In one embodiment, the pharmaceutical composition described herein is administered to a subject in addition to a second pharmaceutical composition containing one or more additional therapeutic agents.

In one embodiment, the two pharmaceutical compositions containing the two different therapies can be administered sequentially or concurrently. In one embodiment, the administration of a second pharmaceutical composition in addition to the pharmaceutical composition described herein can reduce the effective dosage of one or more of the therapies. In one embodiment, the two pharmaceutical compositions may be administered to a subject by the same or different routes of administration.

The pharmaceutical composition of the second therapeutic agent can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

### EXAMPLES

### Example 1

### A Phase I Study of Ganetespib Monotherapy in Treating Solid Tumor and in Combination with Docetaxel in Treating Non-Small Cell Lung Cancer

A total of 3 (three) cohorts are treated in this study. All patients are hospitalized for the duration of Cycle 1, Days 1 through 21.

In the first cohort (Cohort 1), patients with advanced solid tumor malignancies are treated with 75 mg/m² of ganetespib monotherapy. In the second cohort (Cohort 2), patients with advanced solid tumor malignancies are treated with 150 mg/m² of ganetespib monotherapy. In the third cohort (Cohort 3), patients with advanced NSCLC are treated with ganetespib (150 mg/m²) in combination with docetaxel (75 mg/m²). In the case of intolerability of the third cohort's combination regimen, in a fourth cohort (Cohort 4), patients with advanced NSCLC are treated with ganetespib (150 mg/m²) in combination with docetaxel (60 mg/m²).

Each cohort enrolls 3 to 6 patients, depending on toxicity and the inclusion of Cohort 4. A total of 9 patients are enrolled at the recommended combination dose level to better characterize the safety and PK profiles. Each cohort consists of a maximum of 6 evaluable patients, except for the recommended combination dose level, which enrolls a total of 9 patients. Patients who discontinue from the study for reasons other than DLT (dose-limiting toxicity) before completing Cycle 1 are replaced. For all cohorts, patients are hospitalized for the duration of Cycle 1, Days 1 through 21. Dosing of patients proceeds within each cohort according to the following scheme and in accordance with the rules specified above.

Cohorts and Treatments

| | | Ganetespib (mg/m²) | Docetaxel (mg/m²) |
|---|---|---|---|
| Cohort | Indication | Days 1 and 15 | Day 1 |
| 1 | advanced solid | 75 | None |
| | tumor | | |
| | malignancies | | |
| 2 | advanced solid | 150 | None |
| | tumor | | |
| | malignancies | | |
| 3 | advanced NSCLC | 150 | 75 |
| 4 | advanced NSCLC | 150 | 60 |

Cohort 1: Ganetespib Monotherapy 75 mg/m²

The first cohort consists of patients with advanced solid tumor malignancies who receive 75 mg/m² of ganetespib monotherapy on Days 1 and 15 of a 3-week treatment cycle. Treatment continues until disease progression or until occurrence of unacceptable toxicity.

If 1 of 3 patients experiences a DLT at the 75 mg/m² dose level, then up to 3 additional patients are enrolled at this dose level. If ≧2 of the 6 patients experience DLTs or the safety profile is not considered to be acceptable by the Sponsor's Medical Director and Clinical Investigators, further reduction of the dose is considered or the study is terminated. Otherwise, the study proceeds with Cohort 2.

Cohort 2: Ganetespib Monotherapy 150 mg/m²

The second cohort consists of patients with advanced solid tumor malignancies who receive 150 mg/m² of ganetespib monotherapy on Days 1 and 15 of a 3-week treatment cycle. Treatment continues until disease progression or until occurrence of unacceptable toxicity. If 1 of 3 patients experiences a DLT at the 150 mg/m² dose level, then up to 3 additional patients are enrolled at this dose level. If ≧2 of the 6 patients experience DLTs or the safety profile is not considered to be acceptable by the Sponsor's Medical Director and Clinical Investigators, the recommended dose for ganetespib monotherapy is declared as 75 mg/m² and the study is terminated or further adjustment for dose levels in Cohorts 3 and 4 may be considered. Otherwise, 150 mg/m² is declared as the ganetespib dose level to use for further evaluation of a combination regimen with docetaxel and the study proceeds with Cohort 3.

Ganetespib Combination Therapy

Patients with advanced NSCLC, excluding patients with squamous cell histology, are administered ganetespib and docetaxel in Cohort 3.

Cohort 3: Ganetespib Combination Therapy (Docetaxel 75 mg/m²)

The purpose of this cohort is to explore the PK and tolerability of ganetespib in combination with docetaxel. Patients enrolled in this cohort receive ganetespib 150 mg/m² in combination with docetaxel 75 mg/m².

On Day 1 of each 3-week treatment cycle, ganetespib and docetaxel are administered as separate 1-hour intravenous infusions. Administration of ganetespib precedes the administration of docetaxel. There is a 1-hour “rest” period following the end of the ganetespib infusion and prior to docetaxel infusion.

On Day 15, patients receive 150 mg/m² single-agent ganetespib administered via a 1-hour intravenous infusion.

If 1 of 3 patients experiences a DLT at this dose regimen, then up to 3 additional patients are enrolled at the same dose and regimen. If the safety profile in Cohort 3 is not acceptable (e.g., there is more than 1 of 3 patients or ≧2 of 6 patients with DLTs or the safety profile is not considered to be acceptable by the Sponsor's Medical Director and Clinical Investigators), dosing in Cohort 3 is suspended and Cohort 4 is initiated. Otherwise, ganetespib 150 mg/m² in combination with docetaxel 75 mg/m² is declared the dosing regimen in further studies and this cohort resumes enrollment to have a total of 9 patients to better characterize the safety and PK profile for the regimen.

Patients tolerating ganetespib in combination with docetaxel administered on this schedule can continue treatment until disease progression or until occurrence of unacceptable toxicity.

Many Investigators treat second-line advanced NSCLC patients with up to 6 cycles of docetaxel monotherapy. Following completion of 6 cycles of combination therapy (ganetespib and docetaxel), Investigators may decide to continue treatment with ganetespib monotherapy or ganetespib in combination with docetaxel in patients with stable disease (SD) who may receive clinical benefit from continued maintenance treatment. Maintenance therapy may continue until disease progression or until occurrence of unacceptable toxicity.

Cohort 4: Ganetespib Combination Therapy (Docetaxel 60 mg/m²)

If the safety profile in Cohort 3 patients is not acceptable (>1 of 3 patients or ≧2 of 6 patients experience DLTs, or the safety profile is not considered to be acceptable by the Sponsor's Medical Director and Clinical Investigators), a fourth cohort is added with 60 mg/m² dose of docetaxel in combination with ganetespib. Patients treated in this cohort receive ganetespib 150 mg/m² in combination with docetaxel 60 mg/m².

On Day 1 of each 3-week treatment cycle, ganetespib and docetaxel are administered as separate 1-hour intravenous infusions. Administration of ganetespib precedes the administration of docetaxel. There is a 1-hour “rest” period following the end of the ganetespib infusion prior to docetaxel infusion. Ganetespib 150 mg/m² is administered again on Day 15 of each cycle. 6 patients are treated with this dosing regimen.

If 1 of 3 patients experiences a DLT at this dose regimen, then up to 3 additional patients are enrolled at the same dose regimen. If the safety profile in Cohort 4 is not acceptable (>1 of 3 patients or ≧2 of 6 patients experience DLTs or the safety profile is not considered acceptable by the Sponsor's Medical Director and Clinical Investigators), further reduction in the doses may be considered or the study is terminated. Otherwise, the recommended regimen for combination therapy is declared as 150 mg/m² for ganetespib and 60 mg/m² for docetaxel and this cohort resumes enrollment to have a total of 9 patients to better characterize the safety and PK profile for the regimen.

Patients tolerating ganetespib in combination with docetaxel administered on this schedule can continue treatment until disease progression or until occurrence of unacceptable toxicity.

Ganetespib administered is formulated using 90% v/v PEG 300 and 10% v/v Polysorbate 80 at a concentration of 8 mg/mL and was packaged in a Type I glass amber vial, stoppered with a Flurotec®-coated stopper, and sealed. Each vial had a deliverable volume of 12.5 mL (equivalent to 100 mg/vial). The formulation was further diluted with 5% dextrose for injection in infusion container (DEHP-free 500 mL) to a concentration range of 0.02 to 1.2 mg/mL and administered via infusion tubing (DEHP-free) with a 0.22 micron end filter over an hour to the patient. The dosing solution once prepared was administered within 3 hours. In summary, ganetespib monotherapy in patients with advanced solid tumor malignancies is shown to be efficacious and with good safety profile. In addition, ganetespib in combination with docetaxel in patients with advanced non-small-cell lung cancer (NSCLC) of non-squamous histology is also shown to be efficacious.

### Example 2

### A Phase 1b Study of Ganetespib in Combination with Doxorubicin, Cyclophosphamide and Docetaxel in the Treatment of Inflammatory Breast Cancer

This is an open-label, multicenter, Phase 1b, dose-escalation study in patients with locally advanced and inflammatory breast cancer. The purpose of the study is to test the safety and tolerability of ganetespib in patients with operable breast cancer.

Eligible patients must be treatment naïve with histologic confirmation of breast adenocarcinoma, and a tumor that is readily accessible for sequential biopsy. Before the study, patients undergo a complete physical examination, laboratory investigations, chest x-ray, a mammogram and a breast ultrasonography and other imaging studies (e.g., MRI, PET-CT, or mammogram) to determine the local extent of the disease. Abdominal CT scan is performed to exclude metastatic disease. The methods used to document baseline status must be consistently used throughout the study. Prior to entering the study, HER2 status of the tumor is determined by the local lab.

A pharmacodynamics phase consists of ganetespib single agent given at a dose of 150 mg/m² twice for 1 week prior to start of dose escalation phase (approximately on Days −7 and −4). A pre-dose tumor biopsy is obtained within 15+/−3 days prior to initiating treatment with ganetespib. A post-treatment dose is obtained on Day −4, to evaluate the effects of ganetespib on downstream effectors regulating tumor proliferation and survival, as well as potential effects on HIF1-transcriptional activity. Following the biopsy on Day −4, patients start the dose escalation phase. Upon completion of the combination treatment, patients undergo locoregional treatment, as indicated by the local institutional standards

One treatment cycle consists of treatment with ganetespib, anthracycline and cyclophosphamide on Day 1, and ganetespib on Day 15, docetaxel and ganetespib on Day 22, and Ganetespib on Day 37.

The study tests two dose levels of ganetespib with fixed doses of ACT regimen as follows:

Ganetespib is administered on Days 1, 15, 22, 37

Doxorubicin (Adriamycin) is administered at a dose of 60 mg/m² IV on Day 1

Cyclophosphamide (Cytoxan) is administered at a dose of 600 mg/m² IV on Day 1

Docetaxel (Taxotere) is administered at a dose of 75 mg/m² IV on day 22

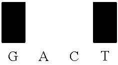

Each treatment cycle is repeated every 6 weeks for up to 3 cyclemonotherapy

Dose escalation proceeds within each cohort according to the following schema:

| | | Ganetespib | | | |
|---|---|---|---|---|---|
| | | [mg/m²] | Doxorubicin | Cyclophosphamide | Docetaxel |
| Dose | | Days 1, 15, | [mg/m²] | [mg/m²] | [mg/m²] |
| Level | N | 22, 37 | Days 1 | Day 1 | Day 22 |
| do-1 | 3-6 | 75 | 60 | 600 | 75 |
| 1 | 3-6 | 100 | 60 | 600 | 75 |
| 2 | 3-6 | 150 | 60 | 600 | 75 |

The starting dose of ganetespib is 100 mg/m² (dose level 1). There must be at least three evaluable patients treated at a dose level before dose escalation can occur. Intra-patient dose escalation is allowed in the study

An evaluable patient is defined as one who has received one cycle of treatment or has experienced a dose limiting toxicity (DLT) after any dose.

If 0 out of 3 patients experience a DLT at 100 mg/m² of ganetespib, enrollment can begin in the next dose level (ganetespib 150 mg/m²).

If 1 out of 3 patients experience a DLT at 100 mg/m² of ganetespib, additional 3 patients are entered at this dose level.

If 1 of these 6 patients experiences a DLT, proceed to dose level 2 (ganetespib 150 mg/m²).

If 2 or more of 6 patients experience a DLT, then dose escalation is stopped, treatment at dose level −1 (ganetespib 75 mg/m²) starts.

The estimated total sample size for this Phase 1 study is approximately 25-30 patients.

Duration of Treatment:

Treatment will continue up to 18 weeks followed by locoregional treatment. Patients, who demonstrate clinical and/or radiological evidence of disease progression at week 12 discontinue treatment and start alternative therapy.

In summary, ganetespib combination therapy in patients with inflammatory breast cancer is shown to be efficacious and with good safety profile.

### Example 3

### A Phase I Study of Ganetespib in Combination with Cisplatin and Pemetrexed, Carboplatin and Pemetrexed, and Carboplatin and Paclitaxel in Patients with Non-Small Cell Lung Cancer

This is a Phase 1, open-label, dose-escalation study in patients with advanced NSCLC with adenocarcinoma histology.

Three cohorts are enrolled:

Cohort 1: patients treated with ganetespib, cisplatin and pemetrexed

Cohort 2: patients treated with ganetespib, carboplatin and pemetrexed

Cohort 3: patients treated with ganetespib, carboplatin and paclitaxel

Treatment Cycle:

In Cohort 1, one treatment cycle consists of treatment with ganetespib, cisplatin and pemetrexed on Day 1, and ganetespib on Day 15

In Cohort 2, one treatment cycle consists of treatment with ganetespib, carboplatin and pemetrexed on Day 1, and ganetespib on Day 15

In Cohort 3, one treatment cycle consists of treatment with ganetespib, carboplatin and paclitaxel on Day 1, and ganetespib on Day 15

Treatment cycles are repeated every 3 weeks. The total planned number of combination cycles is 4. Ganetespib is administered on Days 1 and 15 of each cycle; cisplatin and pemetrexed, carboplatin and pemetrexed, or carboplatin and paclitaxel are administered on Day 1 of each cycle. Each agent is administered as a separate infusion (see Dose-Escalation Table below).

Cohort 1: Cisplatin and Pemetrexed Regimen

On Day 1, ganetespib is administered first, as a 1-hour intravenous (IV) infusion, followed by a rest period of 1 hour. The second infusion is pemetrexed (500 mg/m², IV infusion over 10 minutes). The third infusion is cisplatin (75 mg/m², IV infusion over 2 hours).

On Day 15, ganetespib is administered as a 1-hour IV infusion.

Standard ganetespib premedication is used for both Day 1 and Day 15 infusions.

Cohort 2: Carboplatin and Pemetrexed Regimen

On Day 1, ganetespib is administered first, as a 1-hour IV infusion, followed by a rest period of 1 hour. The second infusion is pemetrexed (500 mg/m², 1-hour IV infusion). The third infusion is carboplatin (AUC 6, IV infusion over 30 minutes).

On Day 15, ganetespib is administered as a 1-hour IV infusion.

Standard ganetespib premedication is be used for both Day 1 and Day 15 infusions.

Cohort 3: Carboplatin and Paclitaxel Regimen

On Day 1, ganetespib is administered first, as a 1-hour IV infusion, followed by a rest period of 1 hour. The second infusion is paclitaxel (200 mg/m², IV infusion over 10 minutes 1 hour). The third infusion is carboplatin (AUC 6, IV infusion over 30 minutes).

On Day 15, ganetespib is administered as a 1-hour IV infusion.

Standard ganetespib premedication is used for both Day 1 and Day 15 infusions. Dose escalation will proceed within each cohort according to the following schema:

| | | | Ganetespib | Pemetrexed | Cisplatin |
|---|---|---|---|---|---|
| | Dose | | [mg/m²] | [mg/m²] | [mg/m²] |
| | Level | N | Days 1 and 15 | Day 1 | Day 1 |
| Cohort 1 | −1 | 3-6 | 75 | 500 | 75 |
| | 1 | 3-6 | 100 | 500 | 75 |
| | 2 | 3-6 | 150 | 500 | 75 |
| | | | Ganetespib | Pemetrexed | |
| | | | [mg/m²] | [mg/m²] | Carboplatin |
| | | N | Days 1 and 15 | Day 1 | Day 1 |
| Cohort 2 | −1 | 3-6 | 75 | 500 | AUC 6 |
| | 1 | 3-6 | 100 | 500 | AUC 6 |
| | 2 | 3-6 | 150 | 500 | AUC 6 |
| | | | Ganetespib | Paclitaxel | |
| | | | [mg/m²] | [mg/m²] | Carboplatin |
| | | N | Days 1 and 15 | Day 1 | Day 1 |
| Cohort 3 | −1 | 3-6 | 75 | 200 | AUC 6 |
| | 1 | 3-6 | 100 | 200 | AUC 6 |
| | 2 | 3-6 | 150 | 200 | AUC 6 |

The starting dose of ganetespib is 100 mg/m² (dose level 1). There must be at least 3 evaluable patients treated at a dose level before dose escalation can occur. An evaluable patient is defined as one who has received one full cycle of treatment or has experienced a DLT after any dose during the first treatment cycle.
If 0 out of 3 patients experience a DLT at 100 mg/m² of ganetespib, enrollment can begin in the next dose level (ganetespib 150 mg/m²).
If >1 of 3 patients experience a DLT at 100 mg/m² of ganetespib, dose escalation is stopped. Treatment at dose level −1 will start (ganetespib 75 mg/m²).
If 1 out of 3 patients experience a DLT at 100 mg/m² of ganetespib, an additional 3 patients are entered at this dose level.
   If 0 of these 3 additional patients experience a DLT, proceed to dose level 2 (ganetespib 150 mg/m²).
   If 1 or more of these 3 additional patients experience a DLT, then dose escalation is stopped, treatment at dose level −1 (ganetespib 75 mg/m²) starts.

Number of Patients: the total sample size for this Phase 1 study (all cohorts, including dose escalation and expansion) is 45-60 patients.

Duration of treatment: the duration of the combination treatment for each regimen is 4 treatment cycles, if tolerated. Patients who complete 4 combination-treatment cycles are evaluated for tumor disease response, general performance status and toxicities. Patients with stable tumor disease or better (CR or PR), good performance status (ECOG 0 or 1) and with mild or no toxicities (NCI CTCAE Grade ≦1) continue maintenance treatment with ganetespib and pemetrexed until unacceptable toxicity or disease progression.

In summary, ganetespib combination therapy in patients with non-small cell lung cancer is shown to be efficacious and with good safety profile.

### Example 4

### A Phase 3 Study of Ganetespib in Combination with Docetaxel in Patients with Triple-Negative Breast Cancer

### Trial Design/Methodology:

This is an open-label, multicenter, randomized Phase 3 study of patients with triple-negative breast cancer (TNBC). Eligible patients are previously untreated for their advanced or metastatic disease and have measurable disease as defined by Response Evaluation Criteria in Solid Tumors (RECIST). Patients are randomized in a 1:1 ratio to receive either ganetespib in combination with docetaxel or docetaxel alone. The study compares the efficacy and tolerability of ganetespib in combination with docetaxel versus docetaxel alone.

The study enrolls approximately 300 patients over a planned 30-month period, and patients are randomized into one of two treatment arms:

Arm A (combination arm): Ganetespib 150 mg/m² in combination with docetaxel 75 mg/m². On Day 1 of each 3-week treatment cycle, ganetespib and docetaxel are administered as separate 1-hour intravenous infusions. Administration of ganetespib precedes the administration of docetaxel. There is a 1-hour “rest” period following the end of the ganetespib infusion prior to docetaxel infusion. Ganetespib 150 mg/m² is administered again on Day 15 of each cycle

Arm B (control arm): Docetaxel 100 mg/m² is administered on Day 1 of a 3 week treatment cycle by 1-hour intravenous infusion

The study is divided into the following phases:

Screening Phase—Screening assessments must occur within 4 weeks of randomization for determination of patient's overall eligibility. These assessments include medical history, ECOG PS, full hematology and biochemistry, serum human chorionic gonadotropin pregnancy test (for all patients of child-bearing potential), radiological assessments of the disease status, demographic information, record of concomitant medication, and record of AEs.

Randomization Phase—Randomization occurs no more than 3 days prior to initiation of treatment. The timing of tumor assessments is based on the randomization date.

Treatment and ganetespib Maintenance Phase—The first dose of study drug is administered within 3 days of randomization. Docetaxel in either treatment arm is administered for a maximum number of cycles according to prevailing practice and investigator decision, generally until disease progression, patient's withdrawal of consent, or intolerability. In Arm B, if the patient comes off treatment due to unacceptable docetaxel-related toxicity, the investigator should continue the patient on ganetespib until disease progression, patient's withdrawal of consent, or unacceptable toxicity. Ganetespib treatment may be continued in patients with radiologic disease progression should the treating physician believe further treatment would provide clinical benefit.

Crossover to treatment with ganetespib in combination with docetaxel or single-agent ganetespib maintenance therapy is not permitted

Follow-Up Phase—Patients are followed up for survival at approximately 6 week intervals

Clinical and laboratory parameters are assessed to evaluate disease status and toxicity in all patients. Safety assessments are performed on all patients at Day 1 and Day 15 of each treatment cycle, including vital signs, complete blood counts, and serum chemistry (including liver function tests). Physical exam, ECOG PS, and body weight are performed pre-dose at Day 1 of each treatment cycle. In both Arm A and Arm B, a 12-lead electrocardiogram (ECG) is performed pre-dose at Day 1 of each treatment cycle. In addition, in Arm A (the combination arm), a 12-lead ECG is performed pre- and post-dose following ganetespib administration at Day 15 of the first 2 treatment cycles. The post-dose ECG is recorded within approximately 60 minutes after the end of the ganetespib infusion or as soon as feasible.

CT/MRI scans of the chest and abdomen are repeated every 6 weeks from the date of last tumor assessment for the first year, and every 12 weeks thereafter until objective disease progression or until beginning a new cancer treatment, whichever is sooner. The same imaging technique must be used throughout the study.

Approximately 300 patients are enrolled in the study.

In summary, ganetespib combination therapy in patients with triple-negative breast cancer is shown to be efficacious and with good safety profile.

### Example 5

### A Phase I/II Study of Ganetespib in Combination with Paclitaxel in Patients with Recurrent, Platinum-Resistant Ovarian, Fallopian Tube or Primary Peritoneal Cancer

A Phase I/II trial of weekly paclitaxel in combination with ganetespib in patients with recurrent, platinum-resistant ovarian, fallopian tube or primary peritoneal cancer is performed. A total of up to 74 patients (up to 18 Phase I patients and up to 56 Phase II patients) with recurrent, platinum-resistant epithelial ovarian, primary peritoneal and fallopian tube carcinoma is enrolled.

Phase I Treatment: Paclitaxel IV given over 1 hour at 80 mg/m² days 1, 8 and 15 of a 28-day cycle. Ganetespib IV at a starting dose of 100 mg/m² on days 1, 8 and 15 of a 28-day cycle. Ganetespib escalation will follow a modified 3+3 design and escalate from 100 mg/m² to 125 mg/m² to 150 mg/m².

Phase II treatment (after MTD): Phase II TPaclitaxel IV given over 1 hour at 80 mg/m² days 1, 8 and 15 of a 28-day cycle. PLUS ganetespib IV at MTD/MED from Phase I on days 1, 8 and 15 of a 28-day cycle.

In summary, ganetespib combination therapy in patients with ovarian, fallopian tube or peritoneal cancer is shown to be efficacious and with good safety profile.

### Example 6

### A Randomized Clinical Study of Ganetespib in p53 Mutant, Metastatic Ovarian Cancer

Each year, approximately 230,000 new cases of ovarian cancer are diagnosed worldwide. Ovarian cancer is the most deadly of the gynecologic cancers, causing approximately 140,000 deaths worldwide each year, including 41,900 deaths in Europe and 14,000 deaths in the US. The serous ovarian cancer subtype, a particularly aggressive form driven by p53 mutations (an HSP90 client protein), makes up 75 to 80% of diagnoses, with approximately 70% of these cases diagnosed in stage III or IV. Platinum-based chemotherapy remains the mainstay of therapy and results in a 5-year survival rate of only 30% and 10% for stages III and IV respectively.

Approximately 70% of advanced ovarian cancers are believed to exhibit mutations in the p53 tumor suppressor gene, which are associated with particularly aggressive, rapid disease progression. Preclinical models have shown that the p53 mutation directly contributes to the acceleration of tumor growth and proliferation. Because the mutation renders the p53 protein unable to fold properly, the chaperone protein Hsp90 is essential to the maintenance and survival of mutant p53 protein. Consequently, there is a pressing need for more effective, innovative treatment strategies to improve survival in this group of epithelial ovarian cancer patients.

A preclinical study demonstrated that inhibition of Hsp90 by ganetespib destroyed the complex between Hsp90 and mutant p53, inducing the degradation of mutant p53 and resulting in potent anti-cancer activity. This activity was substantially stronger in cells with mutant p53 than in other cells. In addition, inhibition of Hsp90 was shown to strongly sensitize mutant p53 cancer cells to treatment with chemotherapies, as has been seen in prior preclinical studies evaluating ganetespib in lung cancer, breast cancer, and other tumor types.

The clinical study aims to achieve this goal by using ganetespib to target what may be a central driver of ovarian cancer aggressiveness and metastatic ability, namely mutant p53. This approach is supported by the preclinical findings as well as encouraging results for ganetespib, including durable objective tumor responses observed with ganetespib monotherapy, in cancers with genetic profiles driven by strong Hsp90 clients, as well as the favorable activity and safety profile observed for the combination of ganetespib and taxanes.

The clinical trial is designed to determine the efficacy of ganetespib and paclitaxel compared to paclitaxel alone in patients with metastatic, p53 mutant, platinum-resistant ovarian cancer. This novel drug strategy is designed to target a central driver of aggressiveness and metastatic ability of epithelial ovarian cancers, namely stabilized mutant p53 protein.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1. A method of treating cancer in a subject, comprising administering an effective amount of ganetespib or a pharmaceutically acceptable salt thereof, in combination with a taxane derivative and a platinum-containing anti-cancer agent.

2. (canceled)

3. The method of claim 2, wherein the platinum-containing agent is cisplatin or carboplatin.

4. The method of claim 2, further comparing an antimetabolite anti-cancer agent.

5. The method of claim 4, wherein the antimetabolite agent is pemetrexed.

6. The method of claim 2, further comprising an alkylating agent.

7. The method of claim 6, wherein the alkylating agent is cyclophosphamide.

8. The method of claim 6, further comprising an anthracycline agent.

9. The method of claim 8, wherein the anthracycline agent is doxorubicin.

10. The method of claim 1, wherein ganetespib is administered at about 75 mg/m², or about 100 mg/m², or about 1²5 mg/m², or about 150 mg/m², or about 175 mg/m², or about 200 mg/m², or about 225 mg/m².

11-12. (canceled)

13. The method of claim 5, wherein pemetrexed is administered at about 300 mg/m², or about 400 mg/m², or about 500 mg/m².

14. The method of claim 3, wherein cisplatin is administered at about 70 mg/m², or about 75 mg/m², or about 80 mg/m².

15. (canceled)

16. The method of claim 1, wherein the cancer is solid tumor, non-small cell lung cancer, breast cancer, ovarian cancer, fallopian tube cancer, prostate cancer, pancreatic cancer, inflammatory breast cancer, or peritoneal cancer.

17. A method of treating cancer with p53 mutation in a subject, comprising administering an effective amount of ganetespib or a pharmaceutically acceptable salt thereof, in combination with a taxane derivative.

18. The method of claim 17 wherein the taxane derivative is docetaxel or paclitaxel.

19. The method of claim 18, wherein the cancer is solid tumor, non-small cell lung cancer, breast cancer, ovarian cancer, prostate cancer, pancreatic cancer, inflammatory breast cancer, bladder cancer, colon cancer, or peritoneal cancer.

20. The method of claim 19, wherein the cancer is metastatic ovarian cancer or triple negative breast cancer (TNBC).

21. The method of claim 20, wherein the metastatic ovarian cancer is platinum-resistant.

22. A pharmaceutical composition comprising an effective amount of ganetespib, an effective amount of an antimetabolite, and an effective amount of a platinum-containing anticancer agent.

23. The composition of claim 22, wherein the antimetabolite is pemetrexed, and the platinum-containing anticancer agent is cisplatin.

24. The composition of claim 23, wherein the antimetabolite is pemetrexed, and the platinum-containing anticancer agent is carboplatin.

25. A pharmaceutical composition comprising an effective amount of ganetespib, an effective amount of a taxane, and an effective amount of a platinum-containing anticancer agent.

26. The composition of claim 25, wherein the taxane is paclitaxel, and the platinum-containing anticancer agent is carboplatin.

27. A pharmaceutical composition comprising an effective amount of ganetespib, an effective amount of a taxane, an effective amount of an anthracycline, and an effective amount of an alkylating anticancer agent.

28. The composition of claim 27, wherein the anthracycline doxorubicin, the alkylating anticancer agent is cyclophosphamide, and the taxane is docetaxel.
